(19) United States
(12) Patent Application Publication
(10) Pub. No.: US 2021/0085396 A1
(43) Pub. Date: Mar. 25, 2021

(54) FOLLICULAR GROWTH INDUCING APPARATUS

(71) Applicants: ST.MARIANNA UNIVERSITY SCHOOL OF MEDICINE, Kanagawa (JP); TERUMO KABUSHIKI KAISHA, Tokyo (JP)

(72) Inventors: Kazuhiro KAWAMURA, Kanagawa (JP); Kazunori TAKAHASHI, Hiroshima (JP); Kazumasa HIROI, Hiroshima (JP)

(21) Appl. No.: 17/109,842

(22) Filed: Dec. 2, 2020

Related U.S. Application Data
(63) Continuation of application No. PCT/JP2019/022319, filed on Jun. 5, 2019.

(30) Foreign Application Priority Data
Jun. 6, 2018 (JP) 2018-109008

(51) Int. Cl. A61B 18/28

(52) U.S. Cl. CPC A61B 18/28; A61B 2018/00559

(57) ABSTRACT

A follicular growth inducing apparatus is suitable for a surgical procedure for inducing follicular growth and is less invasive and less likely to degrade the ovarian functions. The follicular growth inducing apparatus includes an ovarian puncture needle that forms a puncture hole by puncturing an ovary in a traveling direction of ultrasound emitted from a probe in a transvaginal ultrasound device toward the ovary, for inducing growth of follicles, and an optical fiber that guides a laser beam emitted from a laser generator. The ovarian puncture needle includes a needle tube including a basal end, a shaft supported on the basal end, and a needle tip continuous with the shaft. The needle tip punctures the ovary. The optical fiber is installable in the needle tube to have a distal end of the optical fiber adjacent to the needle tip.

### CROSS-REFERENCE TO RELATED APPLICATIONS

This application is a continuation application of International Patent Application No. PCT/JP2019/022319 filed on Jun. 5, 2019, which claims priority to Japanese Patent Application No. 2018-109008 filed on Jul. 6, 2018, the entire contents of which are incorporated by reference.

### BACKGROUND OF INVENTION

### Field of the Invention

The present invention relates to a follicular growth inducing apparatus for inducing the growth of ovarian follicles, and more particularly, to a follicular growth inducing apparatus including an ovarian puncture needle for puncturing an ovarian cortex to physically stimulate the growth of ovarian follicles and an optical fiber installable inside the ovarian puncture needle.

### Background Art

One known cause for female infertility is, for example, polycystic ovarian syndrome (PCOS). PCOS is a disorder that may disable ovulation, with numerous follicles found inside the ovary but having stopped growing, and the ovary itself enlarged and covered with a thick tunica albuginea. PCOS may be treated with a laparoscopic or an open surgical procedure to physically stimulate the ovary and induce the growth of follicles. Specific procedures may include wedge resection in which an ovary is cut in a wedge shape to partially remove the ovarian cortex and laparoscopic ovarian drilling (LOD) in which the ovarian cortex is irradiated with a laser beam to form multiple holes. These procedures may cause natural ovulation or may increase the responsiveness to an administered ovulation inducing drug and cause ovulation.

However, such surgical procedures use general anesthesia, which is highly invasive to the patient and is also expensive. Additionally, LOD can cause death of many follicles due to laser irradiation of the ovarian cortex, possibly degrading the ovarian functions.

Procedures have been developed to at least eliminate general anesthesia, with the techniques described in Patent literatures below.

Patent Literature 1 entitled METHODS AND SYSTEMS FOR THE MANIPULATION OF OVARIAN TISSUES describes a technique for PCOS treatment.

The technique described in Patent Literature 1 will now be described. A system described in Patent Literature 1 includes a) an ovarian tissue apparatus, b) a transvaginal probe including a handle and an ultrasound transducer, and c) a generator configured to supply energy to a therapeutic element. In the system, a) the ovarian tissue apparatus includes a docking device for docking on an ovary, an anchoring member proximate to an ovarian cyst, and a therapeutic element on a distal end of the docking device. The therapeutic element in c) the generator includes an electrode, a cryoablation element, a cooling element, a laser, or a combination of these.

The docking device may be a needle guided by a needle guide, the therapeutic element may be the electrode, and the ultrasound transducer may be the transvaginal probe. The needle can puncture a site near a junction between the ovarian cortex and the ovarian stroma through the needle guide attached to the transvaginal probe, together with the electrode inserted into the junction. The electrode transfers energy to heat and ablate the tissue around the electrode. Such puncturing without general anesthesia is greatly less invasive to the patient, but may form a minimum number of punctures to reduce pain and discomfort, the procedure time, and bleeding.

Patent Literature 2 entitled METHOD AND SYSTEM FOR PHOTO SELECTIVE VAPORIZATION FOR GYNECOLOGICAL TREATMENTS describes an apparatus for photoselective vaporization of tissue of a female reproductive organ.

The technique described in Patent Literature 2 will now be described. The apparatus described in Patent Literature 2 includes a laser to produce laser radiation, and a hysteroscope to direct laser radiation from an optical fiber coupled to the laser and a flow of irrigant to a treatment area on a surface of the tissue. The optical fiber delivers the laser radiation at a wavelength and an irradiance in the treatment area sufficient to vaporize a substantially greater volume of tissue than the volume of residual coagulated tissue caused by the laser radiation.

The target tissue includes uterine tissue, and the target disorder is selected from leiomyoma uteri, rhabdomyoma, endometriosis, endometrial hyperplasia, endometrial cysts, endometrial polyps, menorrhagia, uterine septa, intrauterine adhesions, and cervical intraepithelial neoplasia.

With the above technique, a smaller zone of thermal damage characterized by thermal coagulation remains after the laser irradiation with an increasing volumetric power density, but the rate of vaporization increases. This allows higher levels of laser irradiance while reducing the side effects or thermal damage and allowing a quicker operations.

### CITATION LIST

### Patent Literature

Patent Literature 1: Japanese Unexamined Patent Application Publication (Translation of PCT Application) No. 2018-510012

Patent Literature 2: Japanese Unexamined Patent Application Publication (Translation of PCT Application) No. 2005-518255

Patent Literature 1 describes forming a minimum number of ovarian punctures to reduce the side effects such as bleeding resulting from puncturing. However, LOD, which forms multiple holes in the ovarian cortex, may form ten or more holes depending on the size of the enlarged ovary. Such puncturing of the ovarian cortex may also form at least as many holes as with LOD. In this case, the technique described in Patent Literature 1 can increase bleeding or other side effects and may be inappropriate.

Additionally, the electrode inserted into the junction between the ovarian cortex and the ovarian stroma heats the tissue, possibly causing death of follicles around the electrode and degrading the ovarian functions.

The technique described in Patent Literature 2, which vaporizes uterine tissue by laser radiation, may also cause death of follicles in the ovarian cortex and possibly degrade the ovarian functions in LOD.

### SUMMARY OF INVENTION

In response to the above issue, one or more aspects of the present invention are directed to a follicular growth inducing apparatus for inducing follicular growth that is less invasive without general anesthesia and is suitable for a surgical procedure including forming multiple holes in an ovary with a technique other than laser irradiation and less likely to degrade the ovarian functions.

A follicular growth inducing apparatus according to a first aspect of the present invention includes an ovarian puncture needle that forms a puncture hole for inducing growth of follicles by puncturing an ovary in a traveling direction of ultrasound emitted from a probe in a transvaginal ultrasound device toward the ovary, and an optical fiber that guides a laser beam emitted from a laser generator. The ovarian puncture needle includes a needle tube including a basal end, a shaft supported on the basal end, and a needle tip continuous with the shaft. The needle tip punctures the ovary. The optical fiber is placeable along the ovarian puncture needle to have a distal end of the optical fiber adjacent to the needle tip.

In the above structure, the optical fiber is placeable along the ovarian puncture needle to have, for example, the distal end reaching the needle tip of the ovarian puncture needle. The ovarian puncture needle may be solid or hollow. A solid ovarian puncture needle may receive the optical fiber fastened to its peripheral surface. A hollow ovarian puncture needle may receive the optical fiber in its internal space.

In the above structure, the needle tip of the ovarian puncture needle is inserted into the ovary to form a minute hole while the ovary is being located with ultrasound emitted from the probe in the transvaginal ultrasound device. The tissue surrounding the puncture hole may typically bleed when punctured.

However, the optical fiber placed along the ovarian puncture needle with the distal end adjacent to the needle tip allows the distal end to be either in direct contact with or slightly apart from the surrounding tissue of the puncture hole formed by the needle tip. The bleeding site can thus be precisely irradiated with a laser beam. The irradiation generates heat, which heats the bleeding site to a high temperature to cause the bleeding site to coagulate and thus stop bleeding.

A follicular growth inducing apparatus according to a second aspect of the present invention is the follicular growth inducing apparatus according to the first aspect in which the needle tube has a hollow bore at least in the basal end and the shaft, and the optical fiber is installable in the bore.

The above structure may have the bore in the basal end and the shaft, or in the needle tip, the basal end, and the shaft. In the structure having the bore in the basal end and the shaft, the distal end of the optical fiber comes in contact with the closed needle tip. In the structure having the bore in the needle tip, the basal end, and the shaft, the distal end of the optical fiber extends through the open needle tip. In either structure, the distal end of the optical fiber is adjacent to the needle tip.

The above structure has the effects below in addition to the effects of the first aspect. When the needle tube is inserted into the ovary, the optical fiber inside the bore receives no direct resistance from the surrounding tissue and is thus prevented from slipping off the needle tube.

A follicular growth inducing apparatus according to a third aspect of the present invention is the follicular growth inducing apparatus according to the second aspect further including a connector having a first end connectable to the basal end and a through-hole to communicate with the bore. The optical fiber includes a positioning mark away from the distal end. In the optical fiber installed in the bore through the through-hole, the positioning mark appears in alignment with a second end of the connector.

The above structure has the effects below in addition to the effects of the second aspect. The optical fiber inside the bore or specifically having the distal end adjacent to the needle tip has the positioning mark to appear in alignment with the second end of the connector. Thus, the optical fiber placed in the through-hole in the connector has the distal end adjacent to the needle tip with the positioning mark aligned with the second end of the connector.

A follicular growth inducing apparatus according to a fourth aspect of the present invention is the follicular growth inducing apparatus according to the second aspect further including a connector having a first end connectable to the basal end and a through-hole to communicate with the bore. The optical fiber has a positioning fastener away from the distal end. In the optical fiber installed in the bore through the through-hole, the positioning fastener is engageable with a second end of the connector.

Although the positioning fastener in the apparatus with the above structure may be, for example, a chuck engageable with the second end of the connector having a central hole receiving the optical fiber, the positioning fastener engageable with the second end of the connector may be in other shapes or formed from other materials.

The above structure has the effects below in addition to the effects of the second aspect. The optical fiber inside the bore or specifically having the distal end adjacent to the needle tip can have the positioning fastener engaged with the second end of the connector. Thus, when the optical fiber is placed in the through-hole in the connector, the positioning fastener is engaged with the second end of the connector, thus positioning the distal end adjacent to the needle tip.

A follicular growth inducing apparatus according to a fifth aspect of the present invention is the follicular growth inducing apparatus according to any one of the first to fourth aspects further including a guide being tubular to receive the needle tube, and a fastener that fastens the guide to the probe. The guide is fastened to the probe in a manner elongated in the traveling direction of the ultrasound toward the ovary.

The above structure has the effects below in addition to the effects of any of the first to fourth aspects. The guide is fastened to the probe in a manner elongated in the traveling direction of ultrasound toward the ovary. The needle tube received in the guide is moved farther inside the guide to enter the ovary. This allows the needle tip to reach the ovary in a reliable manner.

A follicular growth inducing apparatus according to a sixth aspect of the present invention is the follicular growth inducing apparatus according to any one of the second to fourth aspects in which the needle tip in the needle tube is closed, the needle tube has the bore in the basal end and the shaft, and the optical fiber is installable in the bore to have the distal end in contact with the closed needle tip.

The above structure has the effects below in addition to the effects of any of the second to fourth aspects. With a laser beam emitted from the laser generator, the optical fiber having the distal end in contact with the closed needle tip heats the needle tip. This can cause the bleeding site to thermally coagulate at the same time as when the puncture hole is formed in the ovary.

A follicular growth inducing apparatus according to a seventh aspect of the present invention is the follicular growth inducing apparatus according to any one of the first to sixth aspects in which the optical fiber includes a photothermal converter at the distal end to convert the laser beam guided by the optical fiber into heat.

In the above structure, the photothermal converter may include a substance that absorbs a laser beam and converts the absorbed energy into heat. The photothermal converter may be, for example, a photothermal conversion layer of the substance on the surface of the optical fiber. The substance may be a titanium compound, such as a titanium dioxide, or carbon.

The above structure has the effects below in addition to the effects of any of the first to sixth aspects. The photothermal converter efficiently generates heat to cause the bleeding site to coagulate more quickly.

### Advantageous Effects

In the structure according to the first aspect, the ovarian puncture needle is used with a transvaginal ultrasound device with local or intravenous anesthesia as used in oocyte retrieval in in vitro fertilization treatment, and can form puncture holes in the ovary without general anesthesia. This structure is less invasive to the patient.

Although puncturing the ovary may cause side effects, or bleeding at the puncture hole, laser irradiation can quickly stop the bleeding and reduce the side effects.

The needle tip of the ovarian puncture needle can form the puncture hole without laser irradiation as in LOD. This structure is unlikely to degrade the ovarian functions.

The follicular growth inducing apparatus according to the first aspect is thus suitable for a surgical procedure for inducing follicular growth and is less invasive without general anesthesia and less likely to degrade the ovarian functions.

The structure according to the second aspect has the advantageous effects below in addition to the advantageous effects of the first aspect. The optical fiber inside the bore is prevented from slipping off the needle tube. This structure allows repeated formation of holes by puncturing, each followed continuously by stopping of the bleeding. The operation thus takes less time and places less burdens on the patient and the operator.

The structure according to the third aspect has the advantageous effects below in addition to the advantageous effects of the second aspect. This structure allows the positioning mark to be aligned with the second end of the connector. The optical fiber having the distal end adjacent to the needle tip can thus transfer heat generated from laser beam irradiation precisely to the bleeding site.

The structure according to the fourth aspect has the advantageous effects below in addition to the advantageous effects of the second aspect. This structure allows the positioning fastener to be engaged with the second end of the connector. The optical fiber having the distal end adjacent to the needle tip can thus be prevented from moving farther than intended or slipping off the needle tube. This structure allows more effective stopping of bleeding.

The structure according to the fifth aspect has the advantageous effects below in addition to the advantageous effects of any of the first to fourth aspects. The needle tube received in the guide is moved farther inside the guide to allow the needle tip to reach the ovary in a reliable manner. This facilitates forming multiple puncture holes regularly across the ovary surface.

The structure according to the sixth aspect has the advantageous effects below in addition to the advantageous effects of any of the second to fourth aspects. The needle tip heated by an emitted laser beam can cause the bleeding site to coagulate thermally at the same time as when a puncture hole is formed in the ovary. This can avoid an urgent laparotomy to stop bleeding in an uncontrolled manner.

The structure according to the seventh aspect has the advantageous effects below in addition to the advantageous effects of any of the first to sixth aspects. The photothermal converter efficiently generates heat to cause the bleeding site to coagulate quickly, thus allowing a more reliable surgical procedure intended for follicular growth induction.

### BRIEF DESCRIPTION OF THE DRAWINGS

FIG. 1 is a plan view of a follicular growth inducing apparatus according to an embodiment in use.

FIG. 2 is a schematic diagram of the follicular growth inducing apparatus according to the embodiment.

FIG. 3A is an enlarged view of a needle tip of a needle tube included in the follicular growth inducing apparatus according to the embodiment and an optical fiber installed in a bore at the needle tip, and FIG. 3B is a cross-sectional view taken along line A-A as viewed in the direction indicated by the arrow in FIG. 3A.

FIG. 4A is an enlarged view of the optical fiber included in the follicular growth inducing apparatus according to the embodiment, and FIG. 4B is a schematic view of a connector included in the follicular growth inducing apparatus.

FIG. 5A is an enlarged view of a connector and a positioning fastener included in a follicular growth inducing apparatus according to a first modification of the embodiment, and FIG. 5B is a cross-sectional view taken along line B-B as viewed in the direction indicated by the arrow in FIG. 5A.

FIG. 6A is an enlarged view of a needle tip of a needle tube included in a follicular growth inducing apparatus according to a second modification of the embodiment, and FIG. 6B is a cross-sectional view taken along line C-C as viewed in the direction indicated by the arrow in FIG. 6A.

### DETAILED DESCRIPTION

### Embodiments

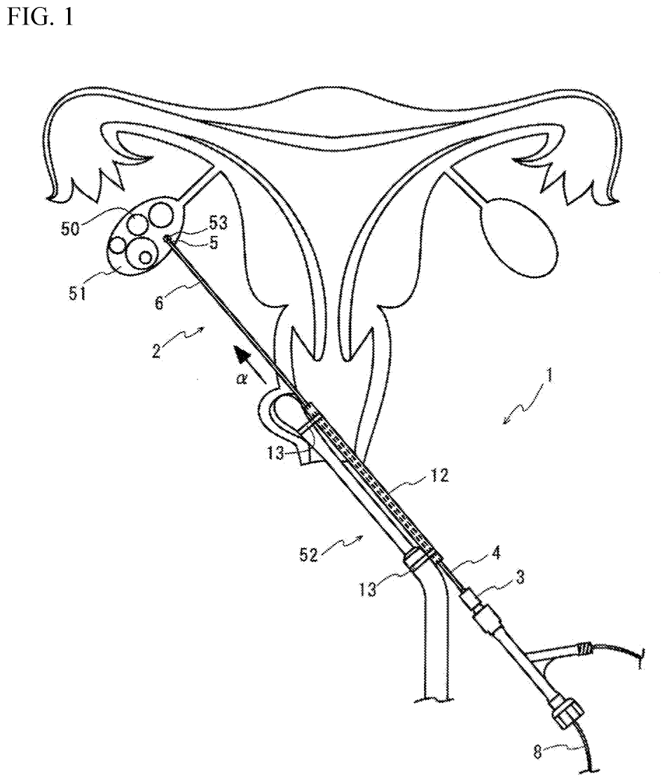

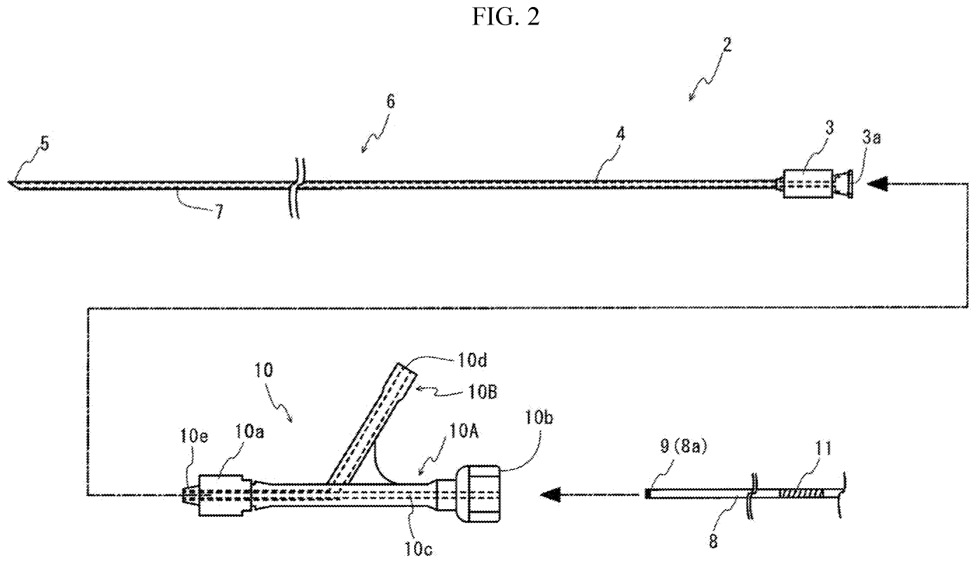

A follicular growth inducing apparatus according to a first embodiment of the present invention will now be described in detail with reference to FIGS. 1 to 6B. FIG. 1 is a plan view of the follicular growth inducing apparatus according to the embodiment in use. FIG. 2 is a schematic diagram of the follicular growth inducing apparatus according to the embodiment.

As shown in FIG. 1, a follicular growth inducing apparatus 1 according the embodiment includes an ovarian puncture needle 2 and an optical fiber 8 to induce the growth of follicles 50. The ovarian puncture needle 2 can form a puncture hole 53 by puncturing an ovary 51 in a traveling direction (indicated by an arrow a in the figure) of ultrasound emitted from a probe 52 included in a transvaginal ultrasound device (not shown) toward the ovary 51. The optical fiber 8 guides a laser beam emitted from a laser generator (not shown). The laser beam is, for example, emitted from a GaAlAs semiconductor laser in the near-infrared region having a wavelength of 808 nm. The optical fiber 8 has a diameter of, for example, 400 μm, but may be another optical fiber.

The follicular growth inducing apparatus 1 further includes a tubular guide 12 receiving a needle tube 6 (described later) included in the ovarian puncture needle 2, and a fastener 13 that fastens the guide 12 to the probe 52. The fastener 13 is specifically a tie band made of resin.

The guide 12 is fastened to the probe 52 in a manner elongated parallel to the length of the probe 52 and in the traveling direction a of the ultrasound toward the ovary 51. The needle tube 6 is thus slidable along the length of the probe 52 in the internal space of the guide 12 that is stably held by the probe 52.

As shown in FIG. 2, the ovarian puncture needle 2 includes the needle tube 6 including a basal end (hub) 3 with a through-hole 3a, a shaft 4 supported on the basal end 3, and a needle tip (bevel) 5 continuous with the shaft 4 and acutely angled to puncture the ovary 51.

In detail, the needle tube 6 has a hollow bore (lumen) 7 that connects the internal spaces of the basal end 3, the shaft 4, and the needle tip 5. The bore 7 receives the optical fiber 8. The needle tube 6 has a diameter of, for example, 18 G, but may have a diameter other than 18 G. The optical fiber 8 is placed along the ovarian puncture needle 2 to position a distal end 8a of the optical fiber 8 adjacent to the needle tip 5. The optical fiber 8 includes a photothermal converter 9 at the distal end 8a to convert the laser beam guided by the optical fiber 8 into heat. The photothermal converter 9 includes a substance that absorbs a laser beam and converts the absorbed energy into heat. The photothermal converter 9 is, for example, a photothermal conversion layer of the substance on the surface of the optical fiber 8. The substance contains titanium dioxide.

The laser beam having a wavelength of 808 nm is less absorbable by water or hemoglobin. The light energy is thus absorbed by the body tissue at a lower rate than being incident and unlikely to generate much heat to allow the body tissue to coagulate sufficiently. The photothermal converter 9 increases the efficiency of the light energy absorption at the distal end 8a and generates heat that allows the body tissue to coagulate sufficiently. When a laser beam having a wavelength other than 808 nm is used to allow the body tissue to sufficiently coagulate thermally, the photothermal converter 9 may be eliminated.

The follicular growth inducing apparatus 1 further includes a connector 10 having a first end 10a connected to the basal end 3 and a through-hole 10c that communicates with the bore 7 in the basal end 3.

The connector 10 includes a body 10A having a second end 10b opposite to the first end 10a across the through-hole 10c, and a side tube 10B connected to the body 10A from near the second end 10b toward the first end 10a. The side tube 10B has a side hole 10d. The side hole 10d is coupled to an irrigant line (not shown) for injecting irrigant for washing the puncture hole 53 formed in the ovary 51. The second end 10b of the body 10A also receives, in addition to the optical fiber 8, a line (not shown) for suctioning the drainage after washing of the puncture hole 53.

The connector 10 has a tapered part 10e protruding from the first end 10a and to be received in the bore 7 of the needle tube 6. The tapered part 10e is fitted in a tapered part located at the basal end 3 and communicating with the bore 7 to connect the basal end 3 and the connector 10.

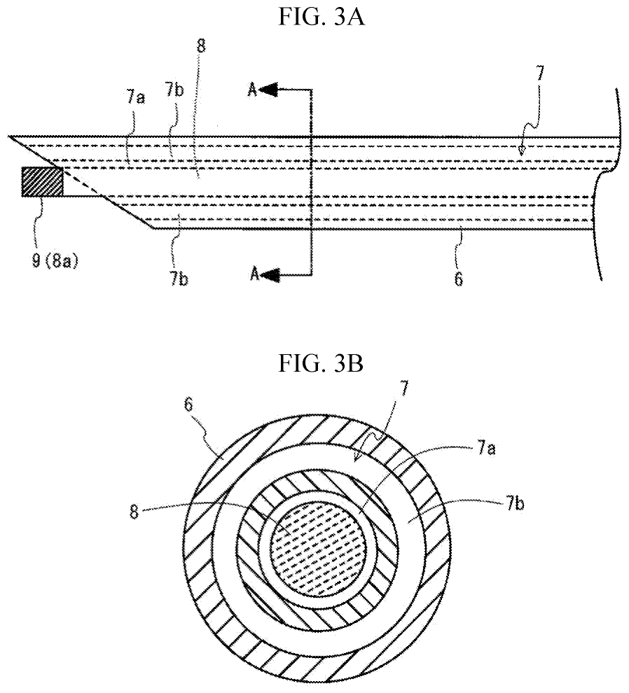

The needle tip of the needle tube included in the follicular growth inducing apparatus according to the embodiment will now be described in detail with reference to FIGS. 3A and 3B. FIG. 3A is an enlarged view of the needle tip of the needle tube included in the follicular growth inducing apparatus according to the embodiment and the optical fiber installed in the bore at the needle tip. FIG. 3B is a cross-sectional view taken along line A-A as viewed in the direction indicated by the arrow in FIG. 3A. In FIGS. 3A and 3B, the components shown in FIGS. 1 and 2 are given the same reference numerals, and will not be described.

As shown in FIGS. 3A and 3B, the needle tube 6 has, in the middle of the bore 7, a narrow tubular drainage suction channel 7a that allows passage of the drainage suctioned after washing. The drainage suction channel 7a and the inner wall of the needle tube 6 define an irrigant injection channel 7b between them to receive injection of irrigant. The drainage suction channel 7a communicates with the through-hole 10c (refer to FIG. 2), and the irrigant injection channel 7b communicates with the side hole 10d (refer to FIG. 2). The needle tube 6 with the above structure receives the optical fiber 8 through the through-hole 10c in the connector 10 and thus receives the optical fiber 8 inside the drainage suction channel 7a.

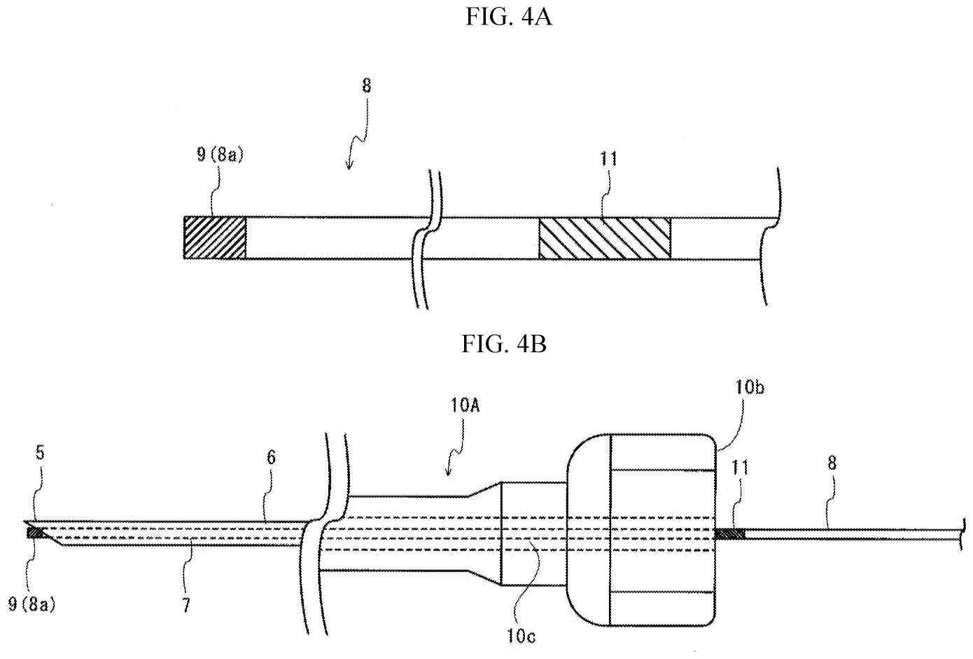

The optical fiber and the connector included in the follicular growth inducing apparatus according to the embodiment will now be described in detail with reference to FIGS. 4A and 4B. FIG. 4A is an enlarged view of the optical fiber included in the follicular growth inducing apparatus according to the embodiment. FIG. 4B is a schematic view of the connector included in the follicular growth inducing apparatus. In FIGS. 4A and 4B, the components shown in FIGS. 1 to 3B are given the same reference numerals, and will not be described.

As shown in FIG. 4A, the optical fiber 8 has a positioning mark 11 away from the distal end 8a. More specifically, the positioning mark 11 includes a piece of tape or a coloring material on the outer circumferential surface of the optical fiber 8.

As shown in FIG. 4B, in the optical fiber 8 installed in the drainage suction channel 7a (refer to FIGS. 3A and 3B) in the bore 7 through the through-hole 10c in the body 10A of the connector 10, the positioning mark 11 has its distal end appearing in alignment with the second end 10b of the body 10A.

In the follicular growth inducing apparatus 1 having the above structure, the guide 12 is preliminarily fastened to the probe 52 with the fastener 13 before the puncture hole 53 is formed in the ovary 51.

The optical fiber 8 is then placed through the through-hole 10c from the second end 10b of the connector 10 toward the needle tube 6 and enters the drainage suction channel 7a (refer to FIGS. 3A and 3B) in the bore 7. The optical fiber 8 is moved farther until the distal end of the positioning mark 11 is aligned with the second end 10b of the connector 10. The optical fiber 8 then has its distal end 8a reaching slightly behind the point of the needle tip 5 of the needle tube 6.

The ovarian puncture needle 2 receiving the optical fiber 8 is then inserted in the guide 12 fastened to the probe 52 placed in the patient's vagina. The needle tip 5 of the needle tube 6 can puncture the surface of the ovary 51 to form the puncture hole 53 while the ultrasound image is being monitored.

At the same time, the laser generator is operated with, for example, a footswitch to emit a laser beam, which is guided to the distal end 8a of the optical fiber 8 and converted into heat by the photothermal converter 9. The distal end 8a positioned slightly behind the point of the needle tip 5 comes in contact with or is slightly apart from the surrounding tissue of the puncture hole 53. The generated heat thus transfers to the surrounding tissue of the puncture hole 53, causing the bleeding site to coagulate and stop bleeding.

Subsequently, a second puncture hole 53 is formed by the needle tip 5 of the needle tube 6 at a position different from the position at which the first puncture hole 53 is formed in the surface of the ovary 51. A laser beam is emitted in the same manner to stop bleeding at the surrounding tissue of the new puncture hole 53. The puncturing followed by stopping of the bleeding is repeated as described above until an intended number of puncture holes 53 is formed.

As described above, the follicular growth inducing apparatus 1 includes the ovarian puncture needle 2 used with the transvaginal ultrasound device without a laparoscope or a laparotomy, and without general anesthesia. The follicular growth inducing apparatus 1 is thus less invasive to the patient and places much less burdens of expenses.

Although puncturing the ovary 51 may cause side effects, or bleeding at the surrounding tissue of the puncture hole 53, the photothermal converter 9 efficiently generates heat to cause the bleeding site to coagulate quickly, thus allowing a more reliable surgical procedure intended for follicular growth induction.

The needle tip 5 of the ovarian puncture needle 2 can form the puncture hole 53 without laser irradiation as in LOD. This structure is unlikely to degrade the ovarian functions.

The follicular growth inducing apparatus 1 is thus suitable for a surgical procedure for inducing follicular growth and is less invasive without general anesthesia and less likely to degrade the ovarian functions.

The follicular growth inducing apparatus 1 also includes the optical fiber 8 installable in the bore 7 without slipping off the needle tube 6. The puncturing followed by stopping of the bleeding is repeated continuously to form multiple puncture holes 53. The operation thus takes less time and places less burdens on the patient and the operator.

The positioning mark 11 is aligned with the second end 10b of the connector 10 to position the distal end 8a of the optical fiber 8 adjacent to the needle tip 5. This allows heat generated by laser beam irradiation to precisely reach the bleeding site.

The guide 12, which is stably held by the probe 52, allows the needle tube 6 to slide in the internal space of the guide 12 along the length of the probe 52. The needle tube 6 moved farther inside the guide 12 can have the needle tip 5 reaching the ovary 51 in a reliable manner. Thus, slightly changing the orientation of the probe 52 allows the multiple puncture holes 53 to be easily formed regularly across the surface of the ovary 51. The ovarian puncture needle 2 may not be constantly held by a hand, thus reducing the operator's fatigue. The guide 12 also reduces accidental damage to internal organs other than the ovary 51.

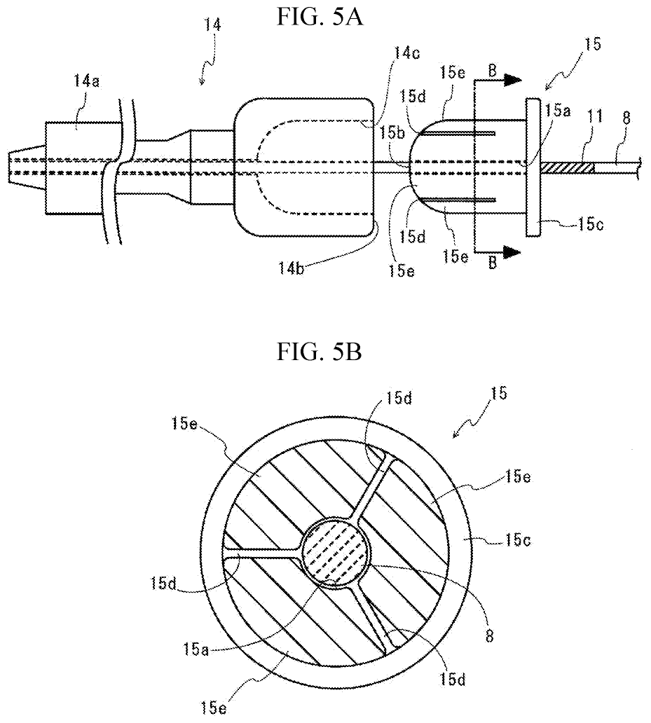

A follicular growth inducing apparatus according to a first modification of the embodiment will now be described with reference to FIGS. 5A and 5B. FIG. 5A is an enlarged view of a connector and a positioning fastener included in the follicular growth inducing apparatus according to the first modification of the embodiment. FIG. 5B is a cross-sectional view taken along line B-B as viewed in the direction indicated by the arrow in FIG. 5A. In FIGS. 5A and 5B, the components shown in FIGS. 1 to 4B are given the same reference numerals, and will not be described.

As shown in FIGS. 5A and 5B, the follicular growth inducing apparatus according to the first modification of the embodiment includes a connector 14, in place of the connector 10 in the follicular growth inducing apparatus 1, and a substantially cylindrical positioning fastener 15 at a position away from the photothermal converter 9 on the optical fiber 8. The positioning fastener 15 is movable relative to the optical fiber 8.

The connector 14 has a first end 14a connected to the basal end 3 of the needle tube 6 and a through-hole 14c that communicates with the bore 7 (refer to FIGS. 2 to 4B) of the needle tube 6. The through-hole 14c is larger in the radial direction of the connector 14 toward a second end 14b of the connector 14 and receives the positioning fastener 15 in a fitted manner. In more detail, the through-hole 14c has a maximum diameter that is equal to the maximum diameter of the positioning fastener 15 excluding a flanged second end 15c.

The positioning fastener 15 is a chuck having a central hole 15a for receiving the optical fiber 8 and is engageable in the through-hole 14c that is open at the second end 14b of the connector 14. The positioning fastener 15 is formed from a flexible rubber material.

As shown in FIG. 5B, the positioning fastener 15 has slits 15d at intervals of 120 degrees about the center of the central hole 15a and defines three segments 15e. The three slits 15d extend from a first end 15b of the positioning fastener 15 without reaching the second end 15c. In the optical fiber 8 installed in the bore 7 through the through-hole 14c in the connector 14, the positioning mark 11 on the optical fiber 8 appears at a position away from the distal end 8a to position the distal end 8a adjacent to the needle tip 5. The other structure of the follicular growth inducing apparatus according to the first modification is the same as the structure of the follicular growth inducing apparatus 1.

The positioning fastener 15 with the above structure receives the optical fiber 8 in the central hole 15a to align the second end 15c of the positioning fastener 15 with the distal end of the positioning mark 11 on the optical fiber 8. The connector 14 with the above structure receives the optical fiber 8 in the through-hole 14c. Subsequently, the positioning fastener 15 is pushed into the through-hole 14c in the connector 14 with the first end 15b ahead until the second end 15c comes in contact with the second end 14b of the connector 14. The three slits 15d in the positioning fastener 15 are then narrowed under pressure in the through-hole 14c, placing the optical fiber 8 inside the central hole 15a in the positioning fastener 15 under pressure from the three segments 15e.

The optical fiber 8 with the distal end 8a adjacent to the needle tip 5 is held immovably with the positioning fastener 15. The other effects of the follicular growth inducing apparatus according to the first modification are the same as the effects of the follicular growth inducing apparatus 1.

The positioning fastener 15 having the above effects can position the distal end 8a of the optical fiber 8 adjacent to the needle tip 5 through simple engagement with the second end 14b of the connector 14 having the above effects. This also prevents the optical fiber 8 from moving farther than intended or slipping off the needle tube 6.

This allows heat generated by the photothermal converter 9 to transfer to the bleeding site in a reliable manner and allows more effective stopping of bleeding. Further, the operator can concentrate on handling the needle tube 6 without worrying about misalignment of the optical fiber 8. The other advantageous effects of the follicular growth inducing apparatus according to the first modification are the same as the advantageous effects of the follicular growth inducing apparatus 1.

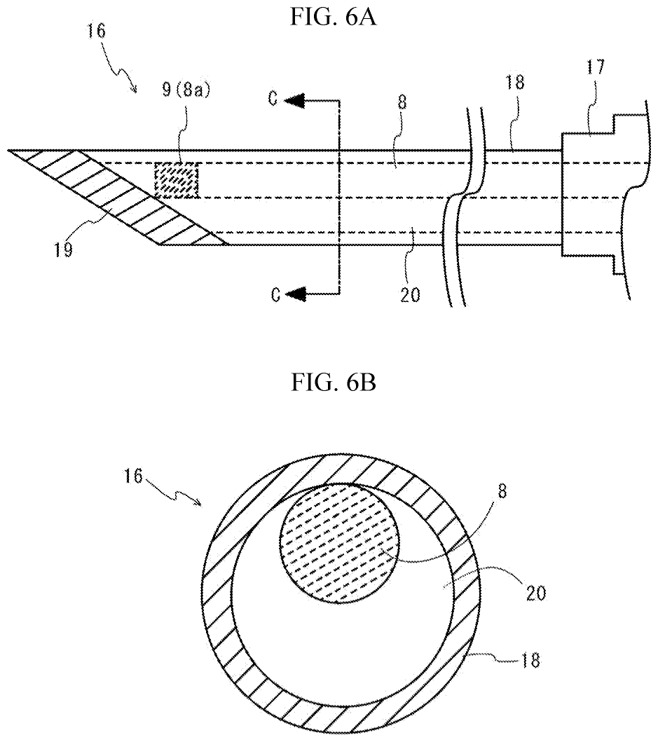

A follicular growth inducing apparatus according to a second modification of the embodiment will now be described with reference to FIGS. 6A and 6B. FIG. 6A is an enlarged view of a needle tip of a needle tube included in the follicular growth inducing apparatus according to the second modification of the embodiment. FIG. 6B is a cross-sectional view taken along line C-C as viewed in the direction indicated by the arrow in FIG. 6A. In FIGS. 6A and 6B, the components shown in FIGS. 1 to 5B are given the same reference numerals, and will not be described.

As shown in FIGS. 6A and 6B, the follicular growth inducing apparatus according to the second modification of the embodiment includes a needle tube 16, in place of the needle tube 6 in the ovarian puncture needle 2 included in the follicular growth inducing apparatus 1.

The needle tube 16 includes a closed needle tip 19 (hatched part) at an acute angle, and a basal end 17 and a shaft 18 with continuous internal spaces defining a bore 20. The needle tube 16 has the closed needle tip 19 and thus does not define the drainage suction channel 7a (refer to FIGS. 3A and 3B) in the bore 20, unlike in the needle tube 6. The optical fiber 8 installed in the bore 20 may thus have the distal end 8a with the photothermal converter 9 in contact with the closed needle tip 19. The connector 10 eliminating the side tube 10B may be used as a connector (not shown) in this modification. The other structure of the follicular growth inducing apparatus according to the second modification is the same as the structure of the follicular growth inducing apparatus 1.

The needle tube 16 with the above structure has the closed needle tip 19 in contact with the distal end 8a of the optical fiber 8, which heats the needle tip 19 with a laser beam emitted from the laser generator. This causes the bleeding site to thermally coagulate at the same time as when the puncture hole 53 is formed in the ovary 51. The other effects of the follicular growth inducing apparatus according to the second modification are the same as the effects of the follicular growth inducing apparatus 1.

The needle tube 16 with the above effects can form the puncture hole 53 in the ovary 51 while causing the bleeding site to thermally coagulate. This can avoid an urgent laparotomy to stop bleeding in an uncontrolled manner. The other advantageous effects of the follicular growth inducing apparatus according to the second modification are the same as the advantageous effects of the follicular growth inducing apparatus 1.

The follicular growth inducing apparatus according to the present invention is not limited to those described in the embodiment and the modifications. For example, the needle tube 6 may be solid without the bore 7. In this example, the optical fiber 8 is fastened to the needle with a fastener. The photothermal converter 9 may be eliminated depending on the wavelength or energy of the laser beam to be used. The cylindrical guide 12 may also be eliminated for certain types of disorder or progression of the symptoms that allow a fewer possible puncture holes 53 to be formed. The connectors 10 and 14 may have shapes different from those described in the embodiments.

### INDUSTRIAL APPLICABILITY

The technique according to one or more embodiments of the present invention may be used for a follicular growth inducing apparatus for inducing growth of follicles.

### REFERENCE SIGNS LIST

1 follicular growth inducing apparatus
2 ovarian puncture needle
3 basal end
3a through-hole
4 shaft
5 needle tip
6 needle tube
7 bore
7a drainage suction channel
7b irrigant injection channel
8 optical fiber
8a distal end
9 photothermal converter
10 connector
10A body
10B side tube
10a first end
10b second end
10c through-hole
10d side hole
10e tapered part
11 positioning mark
12 guide
13 fastener
14 connector
14a first end
14b second end
14c through-hole
15 positioning fastener
15a central hole
15b first end
15c second end
15d slit
15e segment
16 needle tube
17 basal end
18 shaft
19 needle tip
20 bore
50 follicle
51 ovary
52 probe
53 puncture hole

1. A follicular growth inducing apparatus, comprising:
an ovarian puncture needle configured to form a puncture hole by puncturing an ovary in a traveling direction of ultrasound emitted from a probe in a transvaginal ultrasound device toward the ovary, for inducing growth of follicles; and
an optical fiber configured to guide a laser beam emitted from a laser generator,
wherein the ovarian puncture needle includes a needle tube including a basal end, a shaft supported on the basal end, and a needle tip continuous with the shaft, the needle tip is configured to puncture the ovary, and
the optical fiber is placeable along the ovarian puncture needle to have a distal end of the optical fiber adjacent to the needle tip.

2. The follicular growth inducing apparatus according to claim 1, wherein
the needle tube has a hollow bore at least in the basal end and the shaft, and
the optical fiber is installable in the bore.

3. The follicular growth inducing apparatus according to claim 2, further comprising:
a connector having a first end connectable to the basal end and a through-hole to communicate with the bore,
wherein the optical fiber includes a positioning mark away from the distal end, and
in the optical fiber installed in the bore through the through-hole, the positioning mark appears in alignment with a second end of the connector.

4. The follicular growth inducing apparatus according to claim 2, further comprising:
a connector having a first end connectable to the basal end and a through-hole to communicate with the bore,
wherein the optical fiber has a positioning fastener away from the distal end, and
in the optical fiber installed in the bore through the through-hole, the positioning fastener is engageable with a second end of the connector.

5. The follicular growth inducing apparatus according to claim 1, further comprising:
a guide being tubular to receive the needle tube; and
a fastener configured to fasten the guide to the probe,
wherein the guide is fastened to the probe in a manner elongated in the traveling direction of the ultrasound toward the ovary.

6. The follicular growth inducing apparatus according to claim 2, wherein
the needle tip in the needle tube is closed, the needle tube has the bore in the basal end and the shaft, and
the optical fiber is installable in the bore to have the distal end in contact with the closed needle tip.

7. The follicular growth inducing apparatus according to claim 1, wherein
the optical fiber includes a photothermal converter at the distal end to convert the laser beam guided by the optical fiber into heat.
